(19) United States
(12) Patent Application Publication
(10) Pub. No.: US 2017/0232967 A1
(43) Pub. Date: Aug. 17, 2017

(54) PATH DETERMINATION APPARATUS

(71) Applicant: TOYOTA JIDOSHA KABUSHIKI KAISHA, Toyota-shi (JP)

(72) Inventors: Nobuyuki TOMATSU, Toyota-shi (JP); Junichi WAKITA, Nagoya-shi (JP); Ikuma SUZUKI, Okazaki-shi (JP); Akihito NAKAMURA, Toyota-shi (JP); Hiromitsu URANO, Numazu-shi (JP)

(73) Assignee: TOYOTA JIDOSHA KABUSHIKI KAISHA, Toyota-shi (JP)

(21) Appl. No.: 15/429,263

(22) Filed: Feb. 10, 2017

(30) Foreign Application Priority Data
Feb. 16, 2016 (JP) 2016-027158

(51) Int. Cl. B60W 30/18; G05D 1/02; G08G 1/01; G05D 1/00

(52) U.S. Cl. CPC B60W 30/18009; G05D 1/0088; G05D 1/0212; G08G 1/0116; B60W 2520/06; B60W 2720/24; B60W 2550/20; B60W 2900/00

(57) ABSTRACT

A path determination apparatus according to this disclosure includes a gate selecting unit that, when a vehicle is to pass through a tolling station having a plurality of gates, selects a gate to pass through from among the plurality of gates. The gate selecting unit is configured to make a judgement as to whether or not a road which the vehicle travels on until passing through the tolling station is an expressway, as one conditional judgement for selecting the gate to pass through.

### CROSS-REFERENCE TO RELATED APPLICATION

This application is based on and claims the benefit of Japanese Patent Application No. 2016-027158, filed on Feb. 16, 2016, which is incorporated by reference herein in its entirety.

### BACKGROUND

Field of the Disclosure

The present disclosure relates to a path determination apparatus mounted in a vehicle that is capable of autonomous driving.

Background Art

In JP 2001-134895 A, an apparatus is disclosed which, when an own vehicle has approached a tolling station that has a plurality of gates, by causing an ETC on-board device and a car navigation apparatus to conjointly operate, guides the own vehicle to a general gate if an ETC card is not inserted in the ETC on-board device and guides the own vehicle to an ETC gate if an ETC card is inserted in the ETC on-board device.

### SUMMARY OF THE DISCLOSURE

In this connection, tolling stations are provided at a boundary between two different roads, such as between an expressway and an ordinary road, or between one expressway and another expressway. Consequently, when a vehicle that is capable of autonomous driving passes through a tolling station, various changes arise in relation to control, such as the handover of the driving operation accompanying the start or end of autonomous driving, or switching of the system for autonomous driving.

When a plurality of gates are provided at a tolling station, enabling the appropriate selection of a gate for the vehicle to pass through from among the plurality of gates is one task that is required of a path determination apparatus in order to suppress the occurrence of a situation in which control changes adversely affect the travel of the vehicle. In this respect, even if the aforementioned conventional technology is applied to a vehicle that is capable of autonomous driving, a gate will be merely selected according to the presence or absence of an ETC card, and the conventional technology cannot necessarily be said to be sufficient for realizing the appropriate handover of the driving operation or appropriate switching between autonomous driving systems or the like.

The present disclosure has been conceived in consideration of the above described problem, and an object of the present disclosure is to provide a path determination apparatus that, when a vehicle that is capable of autonomous driving passes through a toiling station, can guide the vehicle to an appropriate gate.

A path determination apparatus according to the present disclosure is a path determination apparatus that is mounted in a vehicle which is capable of autonomous driving, and includes a gate selecting unit that, when the vehicle passes through a tolling station having a plurality of gates, selects a gate to pass through from among the plurality of gates. The gate selecting unit is configured to make a judgement as to whether or not a road that the vehicle travels on until passing through the tolling station is an expressway, as one conditional judgement for selecting the gate to pass through. According to this configuration, the vehicle can be guided to an appropriate gate in accordance with the kind of road that the vehicle travels on until passing through a tolling station.

The gate selecting unit may be configured to make a judgement as to whether or not a road that the vehicle is to travel on after passing through the tolling station is an expressway, as one conditional judgement for selecting the gate to pass through. According to this configuration, the vehicle can be guided to an appropriate gate in accordance with the kind of road that the vehicle is to travel on after passing through the tolling station.

The gate selecting unit may also be configured to make a judgement as to whether driving that is performed until the vehicle passes through the tolling station is autonomous driving or manual driving, as one conditional judgement for selecting the gate to pass through. According to this configuration, the vehicle can be guided to an appropriate gate in accordance with the kind of driving that is performed until the vehicle passes through the tolling station.

The gate selecting unit may also be configured to make a judgement as to whether driving to be performed after the vehicle passes through the tolling station is autonomous driving or manual driving, as one conditional judgement for selecting the gate to pass through. According to this configuration, the vehicle can be guided to an appropriate gate in accordance with the kind of driving to be performed after the vehicle passes through the tolling station.

The gate selecting unit may be configured to define a plurality of selection criteria in advance, determine a selection criterion for selecting a gate to be passed through from among the plurality of selection criteria in accordance with the conditional judgement, and select a gate that most satisfies the determined selection criterion from among the plurality of gates. According to this configuration, a gate that most satisfies a condition can be selected based on a selection criterion that is determined by a conditional judgement.

The gate selecting unit may be configured to, when there is a plurality of selection criteria which correspond to a result of the conditional judgement, evaluate each of the plurality of gates based on a priority level of each selection criterion and a level of compatibility of the plurality of gates with each selection criterion, and select a gate for which an evaluation is highest. According to this configuration, even when there is a plurality of selection criteria which correspond to a result of the conditional judgement, a single gate that most suits the condition can be selected.

According to the path determination apparatus of the present disclosure, when a vehicle capable of autonomous driving passes through a tolling station, the vehicle can be guided to an appropriate gate.

### BRIEF DESCRIPTION OF DRAWINGS

FIG. 1 is a schematic block diagram of an autonomous driving apparatus to which a path determination apparatus of an embodiment of the present disclosure is applied;

FIG. 2 is a view for describing a specific example of a gate that satisfies a selection criterion 1;

FIGS. 3A and 3B are views for describing a specific example of a gate that satisfies a selection criterion 2;

FIG. 4 is a view for describing a specific example 1 of a gate that satisfies a selection criterion 3;

FIGS. 5A and 5B are views for describing a specific example 2 of a gate that satisfies a selection criterion 3;

FIG. 6 is a view for describing a specific example 3 of a gate that satisfies a selection criterion 3;

FIG. 7 is a table showing rules for determining the selection criteria in a case of switching from an expressway to an ordinary road at a tolling station;

FIG. 8 is a table showing rules for determining the selection criteria in a case of switching from an ordinary road to an expressway at a tolling station;

FIG. 9 is a table showing rules for determining the selection criteria in a case of switching from one expressway to another expressway at a toiling station;

FIG. 10 is a view in which a main routine for gate selection is represented with a flowchart;

FIG. 11 is a view in which a sub-routine 1 that is called in the main routine is represented by a flowchart;

FIG. 12 is a view in which a sub-routine 2 that is called in the main routine is represented by a flowchart;

FIG. 13 is a view in which a sub-routine 3 that is called in the main routine is represented by a flowchart;

FIG. 14 is a block diagram illustrating a configuration of a path and speed plan creation unit in the autonomous driving apparatus;

FIG. 15 is a table for describing a modified procedure for selecting a gate; and

FIG. 16 is a view in which a modification of the sub-routine 2 that is called in the main routine is represented by a flowchart.

### DETAILED DESCRIPTION

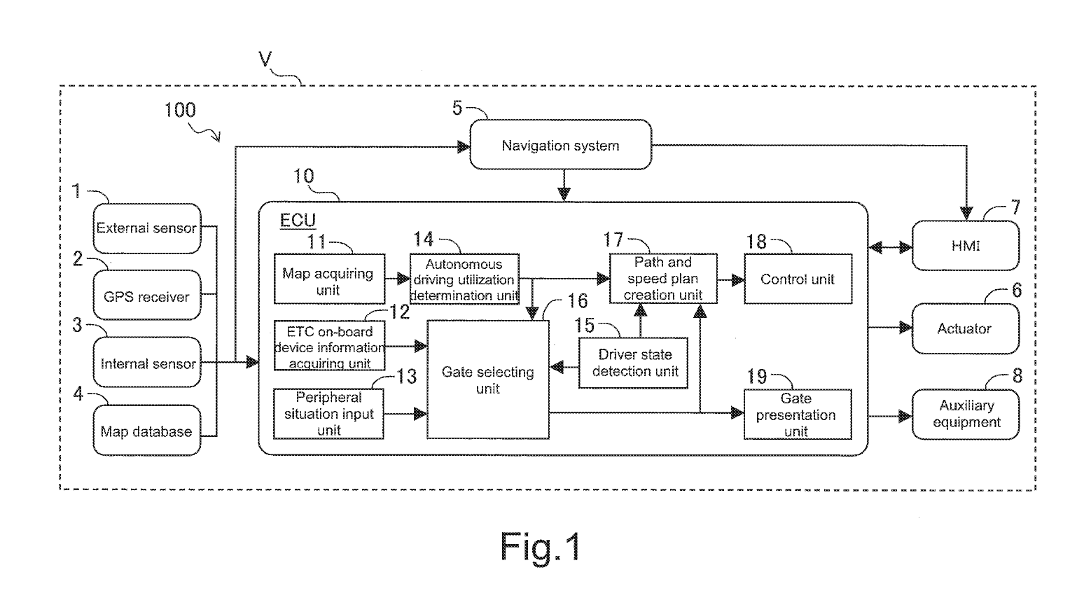

Hereunder, embodiments of the present disclosure are described in detail using the accompanying drawings. As shown in FIG. 1, an autonomous driving apparatus 100 is mounted in a vehicle V such as a passenger vehicle. The autonomous driving apparatus 100 executes autonomous driving of the vehicle V. The term “autonomous driving” refers to driving operations such as acceleration, deceleration and steering of the vehicle V being executed without depending on a driving operation of a driver of the vehicle V. In the autonomous driving apparatus 100 of the present embodiment, a plurality of autonomous driving modes is defined with respect to which the settings of parameters relating to driving operations during autonomous driving are different. The autonomous driving apparatus 100 of the present embodiment executes autonomous driving of the vehicle V using a mode that is selected from among a plurality of autonomous driving modes, and also switches autonomous driving that is being executed to manual driving in accordance with traveling conditions of the vehicle V.

### 1. Configuration of Autonomous Driving Apparatus

AS shown in FIG. 1, the autonomous driving apparatus 100 includes an external sensor 1, a GPS (global positioning system) receiver 2, an internal sensor 3, a map database 4, a navigation system 5, actuators 6, a (human machine interface) 7, auxiliary equipment 8 and an ECU 10.

The external sensor 1 is a detection device that detects the external situation that is information on the surroundings of the vehicle V. Hereinafter, the vehicle V is referred to as “the own vehicle V” to distinguish from other vehicles. The external sensor 1 includes at least one of a camera, radar and LIDER (laser imaging detection and ranging).

The camera is an imaging device that takes images of the external situation of the own vehicle V. The camera is provided, for example, on the vehicle interior side of the front glass of the own vehicle V. The camera may be a monocular camera or may be a stereo camera. A stereo camera has, for example, two imaging units that are disposed so as to reproduce binocular parallax. Information for a depth direction is also included in imaging information of the stereo camera. The camera outputs imaging information relating to the external situation of the own vehicle V to the ECU 10.

The radar utilizes radio waves to detect obstacles outside the own vehicle V. The electric waves are, for example, millimeter waves. The radar transmits radio waves to the surroundings of the own vehicle V, and receives radio waves that are reflected by an obstacle to thereby detect the obstacle. The radar can output, for example, a distance and a direction to the obstacle as obstacle information about the obstacle. The radar outputs the detected obstacle information to the ECU 10. Note that, in a ease of performing sensor fusion, the radar may output received information of the reflected radio waves to the ECU 10.

The LIDER detects obstacles outside the own vehicle V using light. The LIDER sends light to the surroundings of the own vehicle V and receives the light that is reflected by an obstacle, and thereby measures the distance to a reflection point and detects the obstacle. The LIDER can output, for example, a distance and a direction to the obstacle as obstacle information. The LIDER outputs the detected obstacle information to the ECU 10. Note that, in a case of performing sensor fusion, the LIDER may output received information of the reflected light to the ECU 10, Note that, the camera, the LIDER and the radar need not necessarily be provided redundantly.

The UPS receiver 2 receives signals from three or more UPS satellites, thereby acquiring positional information indicating the position of the own vehicle V. For example, a latitude and longitude are included in the positional information. The UPS receiver 2 outputs the measured positional information for the own vehicle V to the ECU 10. Note that other means capable of identifying the latitude and longitude at which the own vehicle V is present may be used instead of the GPS receiver 2.

The internal sensor 3 is a detector which detects information corresponding to a traveling state of the own vehicle V, and information relating to a steering operation amount of a steering operation of the driver of the own vehicle V, and to the state of the driver of own vehicle V. The internal sensor 3 includes at least one of a vehicle speed sensor, an acceleration sensor and a yaw rate sensor in order to detect information corresponding to the traveling state of the own vehicle V. The internal sensor 3 also includes a steering sensor for detecting a steering operation amount, in addition, to detect information relating to the state of a driver who is driving the own vehicle V, the internal sensor 3 includes a driver monitor camera for estimating the state of the driver by image recognition and a biological signal sensor for detecting a biological signal of the driver or the like.

The vehicle speed sensor is a detector which detects the speed of the own vehicle V. As the vehicle speed sensor, for example, a wheel speed sensor is used which is provided at a wheel of the own vehicle V or a drive shaft or the like which rotates integrally with the wheels, and detects the rotational speed of the wheels. The vehicle speed sensor outputs vehicle speed information (wheel speed information) that includes the speed of the own vehicle V to the ECU 10.

The acceleration sensor is a detector which detects the acceleration of the own vehicle V. The acceleration sensor includes, for example, a longitudinal acceleration sensor which detects the acceleration in a longitudinal direction of the own vehicle V, and a lateral acceleration sensor which detects the lateral acceleration of the own vehicle V. The acceleration sensor outputs acceleration information which includes the acceleration of the own vehicle V to the ECU 10.

The yaw rate sensor is a detector which detects a yaw rate (rotational angular velocity) around a vertical axis of the center of gravity of the own vehicle V. As the yaw rate sensor, for example, a gyro sensor is used. The yaw rate sensor outputs yaw rate information which includes the yaw rate of the own vehicle V to the ECU 10.

The steering sensor is a detector that, for example, detects a steering operation amount of a steering operation with respect to a steering wheel by the driver of the own vehicle. A steering operation amount that the steering sensor detects is, for example, a steering angle of the steering wheel or a steering torque with respect to the steering wheel. For example, the steering sensor is provided with respect to a steering shaft of the own vehicle V. The steering sensor outputs information that includes the steering angle of the steering wheel or the steering torque with respect to the steering wheel to the ECU 10.

The driver monitor camera is provided, for example, at a position in front of the driver on a cover of the steering column of the own vehicle, and takes images of the driver. A plurality of driver monitor cameras may be provided so as to take images of the driver from a plurality of directions. The driver monitor camera outputs imaging information about the driver to the ECU 10.

The biological signal sensor is, for example, attached to the driver, or is disposed at a position that is at the periphery of the driver. Signals relating to the heart beat, brain waves, breathing or the like of the human body may be mentioned as examples of the biological signals. Apart from the aforementioned signals, conventional known biological signals can also be used. A biological signal is detected as a waveform that has a period, a frequency and an amplitude. The biological signal sensor outputs the biological signals of the driver to the ECU 10.

When the own vehicle V is a passenger vehicle for a physically handicapped person and a steering operation by the driver of the own vehicle V is performed with respect to a joy stick, the autonomous driving apparatus 100 may include a sensor that detects a steering angle of the joy stick.

The map database 4 is a database that is provided with map information. The map database 4 is formed inside, for example, a HDD (hard disk drive) mounted in the own vehicle V. For example, information regarding kinds of roads, such as whether a road is an expressway or an ordinary road, positional information of roads, information regarding road shapes, and positional information regarding intersections and junctions are included in the map information. For example, classifications of curves and straight portions, as well as the curvatures of curves and the like are included in the information about road shapes. Further, the information relating to expressways includes information on tolling stations provided the entrances to and exits from the expressways, more specifically, information regarding the positions of gates and the like installed at the toiling stations. Further, when the autonomous driving apparatus 100 uses positional information regarding shielding structures such as buildings or walls, or a SLAM (simultaneous localization and mapping) technique, an output signal of the external sensor 1 may be included in the map information. Note that the map database 4 may be stored in a computer of a facility such as an information processing center that is capable of communicating with the own vehicle V.

The navigation system 5 is an apparatus which performs guidance to guide the driver of the own vehicle V to a destination set on a map by the driver of the own vehicle V, The navigation system 5 calculates a route on which the own vehicle V will travel, based on the positional information of the own vehicle V measured by the GPS receiver 2 and the map information of the map database 4. The route may be, for example, a route in which a traffic lane on which the own vehicle V travels is specified in sections which have multiple lanes. The navigation system 5 calculates, for example, a target route from the position of the own vehicle V to the destination, and notifies the target route to the driver by displaying the route on a display and also by audio output from a speaker. The navigation system 5 outputs, for example, information about the target route of the own vehicle V to the ECU 10. The navigation system 5 may use information stored in a computer of a facility such as an information processing center capable of communicating with the own vehicle V. Alternatively, some of the processing which is performed by the navigation system 5 may be performed by a computer at a facility.

The actuators 6 are mechanisms which execute traveling control of the own vehicle V. The actuators 6 include at least a throttle actuator, a brake actuator, and a steering actuator. The throttle actuator controls the amount of air supplied to an engine (degree of throttle opening) according to a control signal from the ECU 10, to thereby control the driving force of the own vehicle V. When the own vehicle V is a hybrid vehicle or an electric vehicle, the throttle actuator is not included, and a control signal from the ECU 10 is input to a motor as a power source, whereby the driving force is controlled.

The brake actuator controls a brake system according to a control signal from the ECU 10, thereby controlling a braking force which is applied to the wheels of the own vehicle V. For example, a hydraulic brake system can be used as the brake system. The steering actuator controls the driving of an assist motor which controls a steering torque of an electric power steering system, in accordance with a control signal from the ECU 10. In this way, the steering actuator controls the steering torque of the own vehicle V.

The HMI 7 is an interface for performing output and input of information between an occupant (including a driver) of the own vehicle V and the driving control apparatus 100. The HMI 7 is provided with, for example, a display panel for displaying image information to an occupant, a speaker for audio output, and operation buttons or a touch panel for allowing the occupant to perform an input operation, and the like. The HMI 7 may perform output of information to an occupant by utilizing a portable information terminal that is wirelessly connected thereto, and may accept an input operation by the occupant that is performed utilizing the portable information terminal.

The auxiliary equipment 8 is equipment which normally can be operated by the driver of the own vehicle V. The auxiliary equipment 8 is a general term for equipment which is not included among the actuators 6. In this case, the auxiliary equipment 8 includes, for example, direction indicator lamps, headlights, wipers, and the like.

The ECU 10 controls driving of the own vehicle V. The ECU 10 is an electronic control unit having a CPU (central processing unit), a RUM (read only memory), a RAM (random access memory), and the like. However, the ECU 10 may be composed of a plurality of electronic control units. In the ECU 10, various functions are realized by loading a program stored in the RUM into the RAM and executing the program with the CPU, A function as the path determination apparatus of the present disclosure is included in the functions realized by the ECU 10. When the ECU 10 functions as a path determination apparatus in the present embodiment, the ECU 10 includes a map acquiring unit 11, an ETC on-board device information acquiring unit 12, a peripheral situation input unit 13, an autonomous driving utilization determination unit 14, a driver state detection unit 15, a gate selecting unit 16, a path and speed plan creation unit 17, a control unit 18 and a gate presentation unit 19. However, the element that is essential as a path determination apparatus is the gate selecting unit 16, and the other elements need not necessarily be included in the path determination apparatus.

The map acquiring unit 11 acquires map information relating to a road that the own vehicle V is traveling on from the map database 4. The acquired map information includes the location of a tolling station, the kind (expressway or ordinary road) and shape of the road up to the tolling station, the kind and shape of a road from the tolling station, and the positions of gates that are installed at the tolling station and the like.

The ETC on-board device information acquiring unit 12 acquires information relating to whether or not an ETC card is inserted in an ETC on-board device (not illustrated). If an ETC card is inserted in the ETC on-board device, the ETC on-board device sends a signal indicating that the ETC card is inserted to the ECU 10. The ETC on-board device information acquiring unit 12 acquires the signal.

The peripheral situation input unit 13 receives information regarding the situation at the periphery of the own vehicle V by the external sensor 1 or by communication. The communication includes inter-vehicle communication which is communication that is performed between the own vehicle V and another vehicle, and road-to-vehicle communication which is communication performed between the own vehicle V and roadside equipment of a road traffic system. The information regarding the situation at the periphery of the own vehicle V that is input includes information regarding moving objects such as pedestrians, other vehicles, motorcycles and bicycles, as well as information regarding stationary objects such as traffic lane lines (white lines, yellow lines), curbs, guard rails, poles, a median strip and building and trees and the like of the road.

The autonomous driving utilization determination unit 14 determines whether or not to utilize the autonomous driving system, based on the map information acquired with the map acquiring unit 11. More specifically, the autonomous driving utilization determination unit 14 determines whether or not to utilize the autonomous driving system until passing through the tolling station based on the information regarding the kind of road and road shape up to the tolling station and the like, and determines whether or not to utilize the autonomous driving system after passing through the tolling station based on information regarding the kind of road and road shape from the tolling station onward and the like. Further, if the autonomous driving system is to be utilized, the autonomous driving utilization determination unit 14 determines which mode to select among a plurality of autonomous driving modes.

The driver state detection unit 15 detects the state of the driver by means of the internal sensor 3. More specifically, the driver state detection unit 15 detects drowsiness, fatigue, inattentiveness or the like by means of image recognition using the driver monitor camera, and detects drowsiness, fatigue, and tension based on biological signals with respect to heartbeat or pulse or the like that are detected by the biological signal sensor. The driver state detection unit 15 can also detect the state of the driver based on information relating to driving operations that is detected based on a steering sensor, an accelerator position sensor and a brake pressure sensor or the like.

The gate selecting unit 16 selects a gate for the own vehicle V to pass through at a tolling station. When the own vehicle V is being driven by autonomous driving, the gate that is selected is a gate to be incorporated into a path plan that is described later, while when the own vehicle V is being driven by manual driving, the gate that is selected is a gate to be suggested to the driver. When a plurality of gates are provided at the tolling station, the gate selecting unit 16 selects one gate to pass through from among the plurality of gates. The selection of a gate is performed based on whether or not the autonomous driving system is to be utilized before and after passing through the tolling station and also the autonomous driving mode in the case of utilizing the autonomous driving system which were determined by the autonomous driving utilization determination unit 14. The selection of a gate is also performed based on information regarding whether or not an ETC card is mounted that was acquired by the ETC on-board device information acquiring unit 12, and information regarding the situation at the periphery of the own vehicle V which was input to the peripheral situation input unit 13. The logic which the gate selecting unit 16 uses to select a gate will be described in further detail later.

The path and speed plan creation unit 17 creates a path and speed plan for the own vehicle V. The path and speed plan includes, for example, the path along which the own vehicle V will proceed on the target route, and the speed, acceleration, deceleration, direction and steering angle and the like of the own vehicle V at each moment. The information used to create the path and speed plan includes the target route calculated by the navigation system 5, map information acquired from the map database 4, whether or not the autonomous driving system is to be utilized before and after passing through the tolling station and also the autonomous driving mode in the case of utilizing the autonomous driving system which were determined by the autonomous driving utilization determination unit 14, the driver state detected by the driver state detection unit 15, and the gate which the own vehicle V is to pass through that was selected by the gate selecting unit 16. The path and speed plan creation unit 17 generates a traveling plan so that the own vehicle V travels in a manner that satisfies criteria such as safety, observance of laws and ordinances, and traveling efficiency on the target route.

The control unit 18 automatically controls the traveling of the own vehicle V based on the path and speed plan generated by the path and speed plan creation unit 17. The control unit 18 outputs control signals in accordance with the path and speed plan to the actuators 6. Thereby, the control unit 18 controls the traveling of the own vehicle V so that autonomous driving of the own vehicle V is executed according to the path and speed plan.

When the own vehicle V approaches a position that is a predetermined distance from the tolling station, the gate presentation unit 19 displays the gate that was selected by the gate selecting unit 16 on the display panel of the HMI 7 and also notifies the driver of the selected gate by voice using the speaker of the HMI 7. If the own vehicle V is being driven by autonomous driving, the gate that is presented through the HMI 7 is the gate that the own vehicle V is scheduled to proceed to thereafter. If the own vehicle V is being driven by manual driving, the gate that is presented through the HMI 7 is the gate to which the own vehicle V should proceed that is suggested to the driver.

### 2. Logic for Gate Selection

### 2-1. Gate Selection Criteria

Next, the logic for gate selection by the ECU 10 as the path determination apparatus will be described. Three selection criteria that are utilized for selecting a gate are defined in advance in the gate selecting unit 16 of the ECU 10. A selection criterion 1 is defined as a criterion that a gate is one at which the driving operation can be safely handed over to the driver. A selection criterion 2 is defined as a criterion that a gate is one at which a smooth transition can be made between different autonomous driving modes. A selection criterion 3 is defined as a criterion that a gate is one through which the destination can be arrived at earliest. The gate selecting unit 16 determines the selection criteria for selecting a gate for the own vehicle V to pass through, and selects a gate that conforms to the thus-determined selection criteria from among the plurality of gates provided at the tolling station.

### 2-1-1. Selection Criterion 1

First, the selection criterion 1 will be described in detail. When the tolling station is a boundary between an expressway and an ordinary road, various changes can occur with respect to the control, such as whether autonomous driving will end at the tolling station or the support level will decrease due to switching of the autonomous driving mode. In this case, it is necessary for the driver to prepare in terms of the attention, awareness, driving posture and the like of the driver, and a handover of the driving operation to the driver in a situation in which the driver is unprepared in all of these ways should be avoided. The selection criterion utilized for selecting a gate in such a case is the selection criterion 1, that is, selection of a gate which enables a safe handover of the driving operation to the driver.

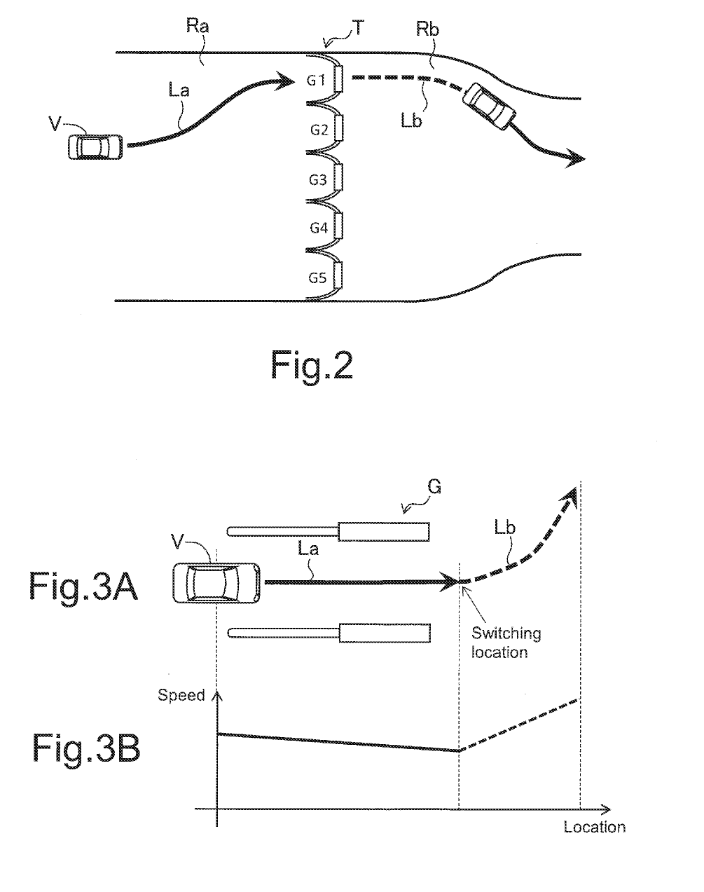

FIG. 2 is a view for describing a specific example of a gate that satisfies the selection criterion 1, In the example illustrated in FIG. 2, a road Ra up to a tolling station T is an expressway, and a road Rb from the tolling station T onward is an ordinary road or an expressway. The width of the road Rh from the tolling station. T onward narrows as the distance from the exit of the tolling station T increases, and eventually becomes the road width of a main road. In the case of such a road shape, the traffic volume is small at the edges of the road Rb in the vicinity of the exit of the tolling station T, and a vehicle is unlikely to interfere with other vehicles even if the vehicle travels at a speed that is low to some extent at those places. Hence, as represented by a path Lb, if the own vehicle V is traveling at an edge of the road Rb, the driver can be given time to prepare to receive the handover of the driving operations before the own vehicle V enters the main road.

In the example illustrated in FIG. 2, five gates G1 to G5 are provided at the tolling station T. The gates which allow a vehicle to travel at an edge of the road Rb after passing through the tolling station T are the gates G1 and G5, and among those two gates the gate G1 is a gate with which a path La that is without a sharp turn can be obtained. Hence, in the example illustrated in FIG. 2, it can be said that the gate G1 is the gate that most satisfies the selection criterion 1.

### 2-1-2. Selection Criterion 2

Next, the details of the selection criterion 2 will be described. Because the degree of difficulty of autonomous driving differs between an expressway and an ordinary road, there is a possibility that different autonomous driving modes will be used for the respective roads. Further, even between one expressway and another expressway, for example, there is a possibility that different autonomous driving modes will be used for the respective expressways due to differences in the road shapes or traffic regulations or the like. In some cases differences with respect to awareness and with respect to the control structure exist between different autonomous driving modes. Therefore, there is a possibility that behavior which is not smooth will occur in the own vehicle V when switching autonomous driving modes. The selection criterion utilized for selecting a gate in this case is the selection criterion 2, that is, a selection criterion that the selected gate is one that enables a smooth transition between different autonomous driving modes.

FIGS. 3A and 3B are views for describing a specific example of a gate that satisfies the selection criterion 2. In the example illustrated in FIG. 3A, paths La and Lb along which the own vehicle V travels before and after passing through a gate G are illustrated in FIG. 3A, and changes in the speed of the own vehicle V in accordance with the traveling position are illustrated in FIG. 3B. In the example illustrated in FIG. 3A, a path La in accordance with an autonomous driving mode until passing through the tolling station is indicated by a solid line, and a path Lb in accordance with an autonomous driving mode after passing through the tolling station is indicated by a broken line. A switching location for switching between the two autonomous driving modes need not be inside the gate G, and may be outside the gate G as shown in FIG. 3A. At this switching location, the path and speed plans of the two autonomous driving modes are connected. If changes in the speed, acceleration, steering angle, angular velocity of the steering angle and the like that arise at such a time are small, an evaluation can be made to the effect that a smooth transition can be made between the different autonomous driving modes.

In the example illustrated in FIG. 3A, a significant change in the traveling direction arises between the path Lb after switching and the path La before switching. This is because the steering angle abruptly changes by a large amount at the switching location. A sudden change in acceleration also occurs at the switching location. Therefore, the gate G through which the own vehicle V passes in the example illustrated in FIG. 3A does not satisfy the selection criterion 2. The gate at which changes in the speed, acceleration, steering angle, angular velocity of the steering angle and the like between before and after switching can be most suppressed, that is, the gate at which changes in the behavior of the own vehicle V can be most suppressed is selected as the gate that most satisfies the selection criterion 2.

Further, gates which do not cause a situation to arise which the autonomous driving mode is unable to cope with after passing through the tolling station are also included in the gates that satisfy the selection criterion 2. For example, in some cases a road up to a tolling station is an ordinary road and a road after a tolling station is an expressway. In such a case, when an autonomous driving mode that is executed on the expressway is not compatible with a pedestrian identification, a gate at which a pedestrian is not close to is selected as a gate that satisfies the selection criterion 2. The situation in the vicinity of the gate may be acquired by the external sensor 1 of the own vehicle V, or may be acquired by inter-vehicle communication or road-to-vehicle communication.

### 2-1-3. Selection Criterion 3

Next, the details of the selection criterion 3 will be described. In some cases a road branches immediately after passing through a tolling station. In such a case, depending on the positional relation between the target route after passing through the tolling station and the gate that is passed through, there is a possibility that it will take time to pass through a junction because the degree of difficulty of the driving operation increases. The selection criterion utilized to select a gate in such a case is the selection criterion 3, that is, a selection criterion that a gate is selected which allows the destination to be reached earliest.

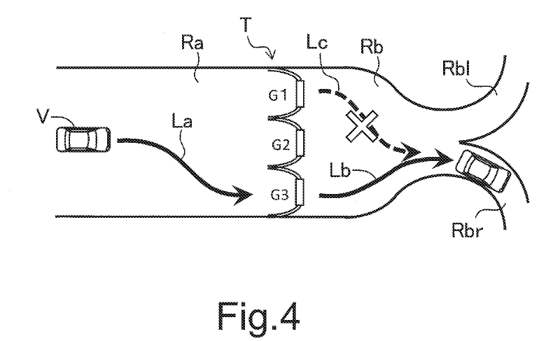

FIG. 4 is a view for describing a specific example 1 of a gate that satisfies the selection criterion 3. In the example illustrated in FIG. 4, a tolling station T at which three gates G1 to G3 are provided is installed at the front of a road. Ra on which the own vehicle V is traveling. A road Rb from the tolling station T onward branches into two roads Rb1 and Rbr immediately after the tolling station T. In this case it is assumed that the road Rbr on the right side in the direction in which the own vehicle V is proceeding is set as the target route. In this case, if the own vehicle V attempts to enter the road Rbr from a gate G1 that is on the leftmost side as indicated by a path Lc, if there is a large amount of traffic it will be necessary for the own vehicle V to cut across the path of other vehicles or to wedge in front of other vehicles. Consequently, if the gate G1 is selected, not only will time be used wastefully to pass through the junction, but the own vehicle V will also disturb the flow of traffic and cause inconvenience for other vehicles.

On the other hand, when the own vehicle V is guided to the gate G3 that is on the rightmost side as indicated by a path La and enters the road Rbr from the gate G3 on the rightmost side as indicated by a path Lb, the own vehicle V will not cut across the path of other vehicles or wedge in front of other vehicles. Hence, in the example illustrated in FIG. 4, the gate that most satisfies the selection criterion 3 is the gate G3, and by selecting the gate G3 it is possible for the own vehicle V to enter the road Rbr in the shortest time. That is, when a road branches immediately after the tolling station T, a gate that is in the same direction as the road that is set as the target route is selected as the gate that most satisfies the selection criterion 3.

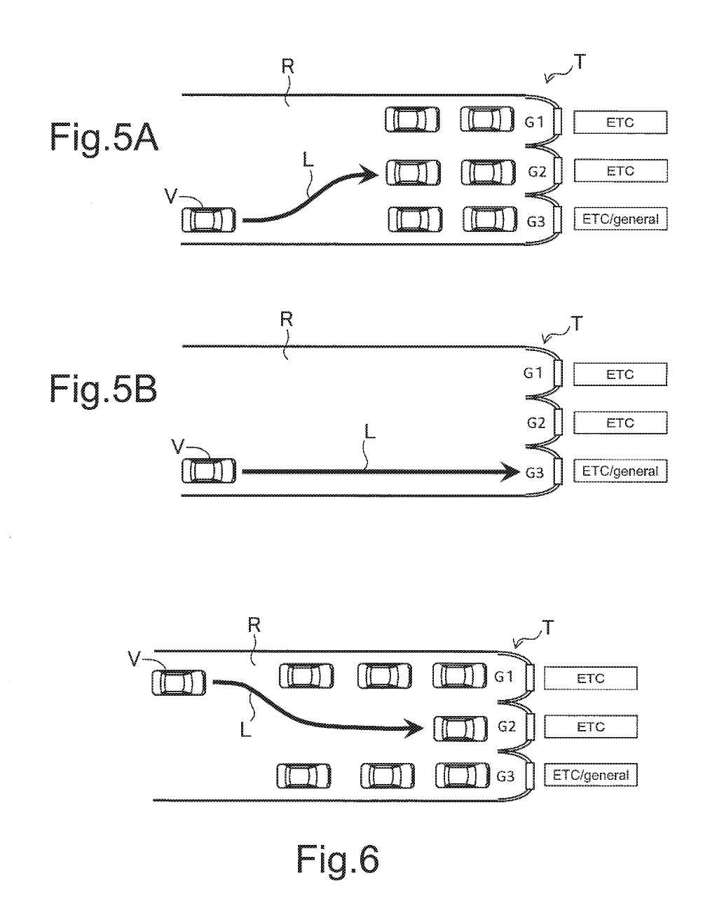

FIG. 5 is a view for describing a specific example 2 of a gate that satisfies the selection criterion 3. In the example illustrated in FIG. 5, a tolling station T at which three gates G1 to G3 are provided is installed at the front of a road R on which the own vehicle V is traveling. The gate G1 on the leftmost side and the gate G2 at the center are gates that are provided in dedicated ETC lanes. The gate G3 on the rightmost side is a gate that is provided in a dual ETC/general lane. When the own vehicle V can utilize ETC (more specifically, when an ETC on-board device is mounted in the own vehicle V, and the ETC on-hoard device is not broken and the time limit for the ETC card has not expired), there is a high probability that the own vehicle V can pass through the tolling station T faster by proceeding to a dedicated ETC lane. However, even when the own vehicle V can utilize ETC, if the dual ETC/general lane is empty, there are also eases where the own vehicle V can pass through the toiling station T faster by proceeding to the dual ETC/general lane.

In example illustrated in FIG. 5A, the same number of other vehicles are in line at the three gates G1 to G3. In this case, the gate which the own vehicle V should proceed to is the gate G1 or gate G2 at which the dedicated ETC lanes are provided, and among those the gate G2 is the gate with which a path L which has less of a sharp turn can be obtained. Therefore, in example illustrated in FIG. 5A, it can be said that the gate G2 most satisfies the selection criterion 3. On the other hand, in example illustrated in FIG. 5B, other vehicles are not lined up at any of the three gates G1 to G3, In this case, the gate which the own vehicle V should proceed to is the gate G3 for which there is no change in direction of the path L and which is nearest to the own vehicle V. Therefore, in example illustrated in FIG. 5B, it can be said that the gate that most satisfies the selection criterion 3 is the gate G3. However, in example (A), if it is known through inter-vehicle communication or road-to-vehicle communication that the other vehicles which are lined up at the gate G3 are all vehicles which can utilize ETC, the gate to which the own vehicle V should proceed is the gate G3 that is nearest to the own vehicle V. That is, in this case, the gate G3 is the gate that most satisfies the selection criterion 3.

FIG. 6 is a view for describing a specific example 3 of a gate that satisfies the selection criterion 3. In the example illustrated in FIG. 6, a tolling station T at which three gates G1 to G3 are provided is installed at the front of a road R on which the own vehicle V is traveling. The gate G1 on the leftmost side and the gate G2 at the center are gates that are provided in dedicated ETC lanes. The gate G3 on the rightmost side is a gate that is provided in a dual ETC/general lane. When the road is busy, lines of other vehicles form in front of the respective gates G1 to G3. In such a case, there is a high probability that the tolling station T can be passed through more quickly if the own vehicle V proceeds to the gate with the shortest line of other vehicles. The length of a line of other vehicles can be ascertained by detecting the tail end of the line by means of the external sensor. In the example illustrated in FIG. 6, since the gate with the shortest line of other vehicles is the center gate G2, the gate that most satisfies the selection criterion 3 is the gate G2.

The above described three specific examples are not exclusive, and it is also possible for the above described three specific examples to overlap depending on the road shape or traffic situation. In such a case, an overall determination is made and a gate through which the own vehicle V can arrive earliest at the destination is selected as the gate that most satisfies the selection criterion 3.

### 2-2. Determination of Selection Criterion

The gate selecting unit 16 determines a selection criterion to be utilized for selecting the gate for the own vehicle V to pass through based on the above described three selection criteria 1 to 3. The selection criterion is decided in accordance with a combination of the following four conditions:

Condition 1: whether the road that the own vehicle V travels on until passing through the tolling station is an expressway or an ordinary road

Condition 2: whether the road that the own vehicle V travels on after passing through the tolling station is an expressway or an ordinary road

Condition 3: whether driving that is performed until the own vehicle V passes through the tolling station is autonomous driving or manual driving

Condition 4: whether driving that will be performed after the own vehicle V passes through the tolling station is autonomous driving or manual driving

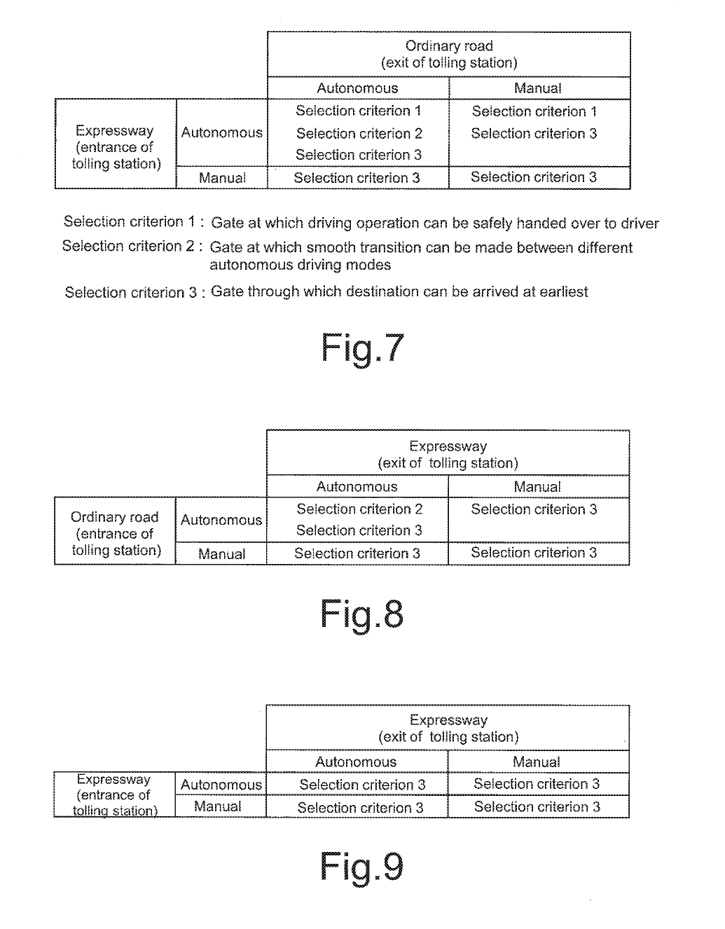

Rules for determining the selection criteria based on a combination of the above four conditions can be expressed in the tables shown in FIGS. 7 to 9.
2-2-1. Selection Criteria Determination Rule 1/from Highway to Ordinary Road

FIG. 7 is a table showing rules for determining the selection criteria in a case of switching from an expressway to an ordinary road at a tolling station. As shown in the table, when the driving performed on the expressway is autonomous driving and the driving performed on the ordinary road is also autonomous driving, the selection criteria to be utilized are the selection criteria 1, 2 and 3. When the driving performed on the expressway is autonomous driving and the driving performed on the ordinary road is manual driving, the selection criteria to be utilized are the selection criteria 1 and 3. Note that, in cases such as these in which there are a plurality of selection criteria that can be utilized, any one of the plurality of selection criteria may be utilized exclusively, or all of the selection criteria may be utilized simultaneously (a specific method for simultaneous utilization will be described later). When the driving performed on the expressway is manual driving and the driving performed on the ordinary road is autonomous driving, the selection criterion to be utilized is the selection criterion 3. When the driving performed on the expressway is manual driving and the driving performed on the ordinary road is also manual driving, the selection criterion to be utilized is the selection criterion 3.

2-2-2. Selection Criteria Determination Rule 2/from Ordinary Road to Highway

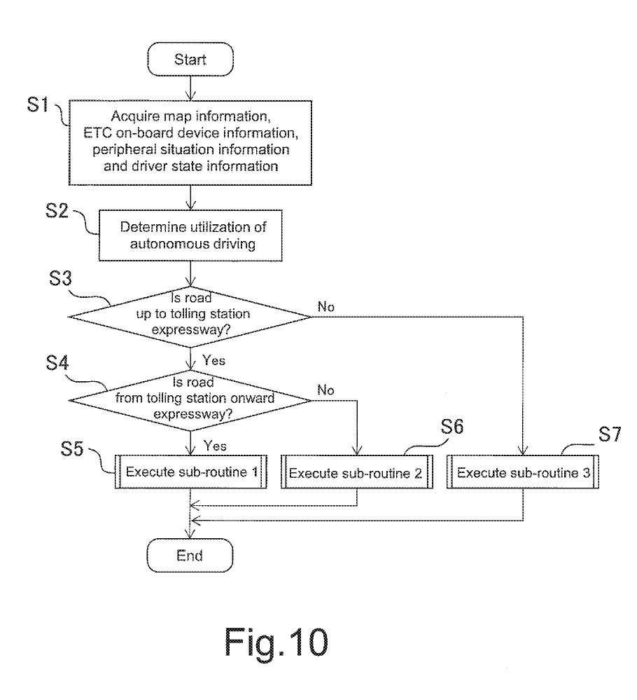

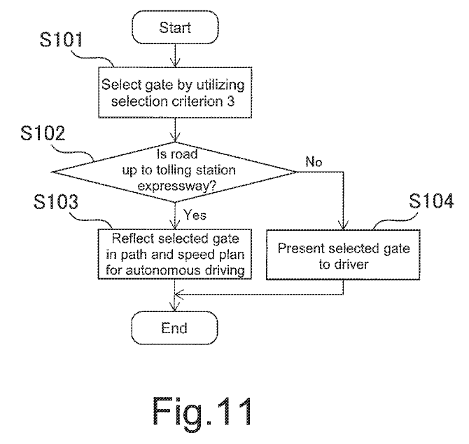

FIG. 8 is a table showing rules for determining the selection criteria in a case of switching from an ordinary road to an expressway at a tolling station. As shown in the table, when the driving performed on the ordinary road is autonomous driving and the driving performed on the expressway is also autonomous driving, the selection criteria to be utilized are the selection criteria 2 and 3. When the driving performed on the ordinary road is autonomous driving and the driving performed on the expressway is manual driving, the selection criterion to be utilized is the selection criterion 3. When the driving performed on the ordinary road is manual driving and the driving performed on the expressway is autonomous driving, the selection criterion to be utilized is the selection criterion 3. When the driving performed on the ordinary road is manual driving and the driving performed on the expressway is also manual driving, the selection criterion to be utilized is the selection criterion 3.

2-2-3. Selection Criteria Determination Rule 3/from Highway to Another Highway

FIG. 9 is a table showing rules for determining the selection criteria in a case of switching from one expressway to another expressway at a tolling station. When switching from one expressway to another expressway, in comparison to switching from an expressway to an ordinary road or switching from an ordinary road to an expressway, handover of the driving operation is comparatively easy, and it is also easy to smoothly perform a transition between different autonomous driving modes. Hence, as shown in the table, when switching from one expressway to another expressway, the selection criterion that is utilized when switching from autonomous driving to autonomous driving, from autonomous driving to manual driving, from manual driving to autonomous driving, or from manual driving to manual driving is, in each case, the selection criterion 3.

### 2-3. Gate Selection Procedures

The gate selecting unit 16 selects a gate for the own vehicle V to pass through based on the above described rules. Selection of a gate is performed in accordance with predetermined procedures. The procedures for gate selection are described in a gate selection program stored in the ROM. The gate selection program is constituted by a main routine that is executed at a predetermined timing (for example, a time point at which the own vehicle V approaches a position that is a predetermined distance from the tolling station), and three sub-routines that are called in the main routine.

### 2-3-1. Details of Main Routine

FIG. 10 is a view in which the main routine for gate selection is represented with a flowchart. Hereunder, the contents of each step constituting the main routine are described in order from step S1.

In step S1, the ECU 10 acquires map information, ETC on-board device information, peripheral situation information and driver state information that are information required for making a gate selection from the map acquiring unit 11, the ETC on-board device information acquiring unit 12, the peripheral situation input unit 13 and the driver state detection unit 15, respectively.

In step S2, by means of the autonomous driving utilization determination unit 14, the ECU 10 determines whether or not to utilize the autonomous driving system until passing through a tolling station, whether or not to utilize the autonomous driving system after passing through the tolling station, and if the autonomous driving system is to be utilized, which autonomous driving mode to utilize.

In step S3, the gate selecting unit 16 of the ECU 10 performs a conditional judgement regarding whether or not a road that the own vehicle V travels on until passing through the tolling station is an expressway. This conditional judgement is performed based on the map information acquired in step S1. If the result of the conditional judgement is “Yes” (when the road that the own vehicle V travels on until passing through the tolling station is an expressway), the present routine proceeds to step S4. If the result of the conditional judgement is “No” (when the road that the own vehicle V travels on until passing through the tolling station is an ordinary road), the present routine proceeds to step S7.

In step S4, the gate selecting unit 16 performs a conditional judgement for determining whether or not the road that the own vehicle V will travel on after passing through the tolling station is an expressway. This conditional judgement is performed based on the map information acquired in step S1. If the result of the conditional judgement is “Yes” (when the road that the own vehicle V will travel on after passing though the tolling station is an expressway), the present routine proceeds to step S5. If the result of the conditional judgement is “No” (when the road that the own vehicle V will travel on after passing through the tolling station is an ordinary road), the present routine proceeds to step S6.

In the main routine, finally the processing of any one of steps S5, S6 and S7 is alternatively selected. If the processing in step S5 is selected, a sub-routine 1 that is described later is called and executed. If the processing in step S6 is selected, a sub-routine 2 that is described later is called and executed. If the processing in step S7 is selected, a sub-routine 3 that is described later is called and executed.

### 2-3-2. Details of Sub-Routine 1

The sub-routine 1 is called in the main routine when a road that the own vehicle V travels on until passing through the tolling station is an expressway and a road that the own vehicle V will travel on after passing through the tolling station is also an expressway. FIG. 11 is a view in which the sub-routine 1 is represented by a flowchart. Hereunder, the contents of each step constituting the sub-routine 1 will be described in order from step S101.

In step S101, the gate selecting unit 16 utilizes the selection criterion 3 to select a gate from among the plurality of gates provided at the tolling station. That is, the gate selecting unit 16 selects the gate through which the destination can be reached earliest as the gate for the own vehicle V to pass through.

In step S102, the gate selecting unit 16 performs a conditional judgement to judge whether driving that will be performed until the own vehicle V passes through the toiling station is autonomous driving or manual driving. This conditional judgement is performed based on the autonomous driving utilization determination performed in step S2 of the main routine. If the result of the conditional judgement is “Yes” (when the driving performed until the own vehicle V passes through the tolling station is autonomous driving), the present routine proceeds to step S103. If the result of the conditional judgement is “No” (when the driving that is performed until the own vehicle V passes through the tolling station is manual driving), the present routine proceeds to step S104.

In step S103, the gate selecting unit 16 outputs the gate selected in step S102 to the path and speed plan creation unit 17 and the gate presentation unit 19. The path and speed plan creation unit 17 reflects the gate selected by the gate selecting unit 16 in the path and speed plan for autonomous driving. The gate presentation unit 19 presents the gate selected by the gate selecting unit 16 to the driver through the HMI 7.

In step S104, the gate selecting unit 16 outputs the gate selected in step S102 to the gate presentation unit 19. The gate presentation unit 19 presents the gate that the gate selecting unit 16 selected to the driver through the HMI 7, to suggest the gate to which the own vehicle V should proceed to the driver.

### 2-3-3. Details of Sub-Routine 2

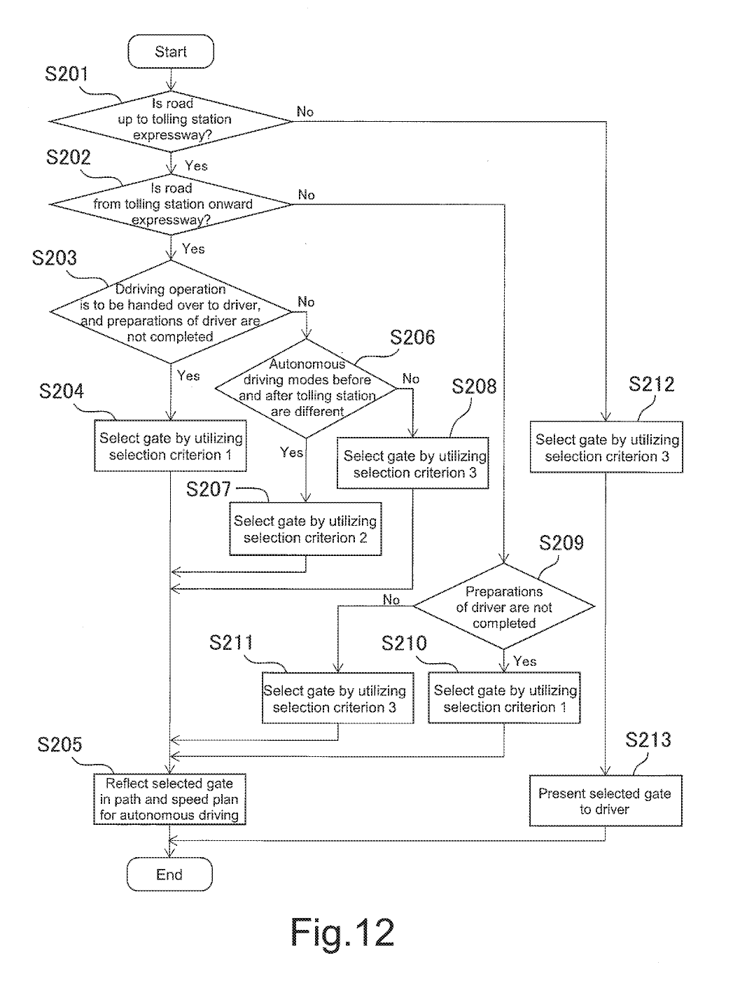

The sub-routine 2 is called in the main routine when a road that the own vehicle V travels on until passing through the tolling station is an expressway and a road that the own vehicle V will travel on after passing through the tolling station is an ordinary road. FIG. 12 is a view in which the sub-routine 2 is represented by a flowchart. Hereunder, the contents of each step constituting the sub-routine 2 will be described in order from step S201.

In step S201, the gate selecting unit 16 performs a conditional judgement to judge whether driving that will be performed until the own vehicle V passes through the tolling station is autonomous driving or manual driving. This conditional judgement is performed based on the autonomous driving utilization determination performed in step S2 of the main routine. If the result of the conditional judgement is “Yes” (when the driving performed until the own vehicle V passes through the toiling station is autonomous driving), the present routine proceeds to step S202. If the result of the conditional judgement is “No” (when the driving that is performed until the own vehicle V passes through the tolling station is manual driving), the present routine proceeds to step S212.

In step S202, the gate selecting unit 16 performs a conditional judgement to judge whether driving that will be performed after the own vehicle V passes through the tolling station is autonomous driving or manual driving. This conditional judgement is performed based on the autonomous driving utilization determination performed in step S2 of the main routine. If the result of the conditional judgement is “Yes” (when the driving that will be performed after the own vehicle V passes through the tolling station is autonomous driving), the present routine proceeds to step S203. If the result of the conditional judgement is “No” (when the driving that will be performed after the own vehicle V passes through the tolling station is manual driving), the present routine proceeds to step S209.

In step S203, the gate selecting unit 16 performs a conditional judgement to judge whether or not both a first condition that a part of the driving operation is to be handed over to the driver and a second condition that preparations of the driver (preparations to receive handover of the driving operations) are not completed are established. Whether or not the first condition is established is determined based on the autonomous driving utilization determination performed in step S1 of the main routine. Whether or not the second condition is established is determined based on the driver state that is acquired in step S1 of the main routine. If the result of the conditional judgement in step S203 is “Yes” (when it is desired to handover a part of the driving operation to the driver, but the preparations of the driver are not completed), the present routine proceeds to step S204. If the result of the conditional judgement in step S203 is “No” (when a part of the driving operation is not to be to handed over to the driver, or when the preparations of the driver are completed), the present routine proceeds to step S206.

In step S204, the gate selecting unit 16 utilizes the selection criterion 1 to select a gate from among the plurality of gates provided at the tolling station. That is, a gate which enables the driving operation to be safely handed over to the driver is selected as the gate for the own vehicle V to pass through. After the processing in step S204, the present routine proceeds to step S205.

In step S206, the gate selecting unit 16 performs a conditional judgement to judge whether or not the autonomous driving mode utilized after passing through the tolling station will be different from the autonomous driving mode utilized before passing through the tolling station. This conditional judgement is performed based on the autonomous driving utilization determination performed in step S2 of the main routine. If the result of the conditional judgement is “Yes” (when the autonomous driving mode after passing through the tolling station is different from the autonomous driving mode before passing through the tolling station), the present routine proceeds to step S207. If the result of the conditional judgement is “No” (when the autonomous driving mode after passing through the tolling station and the autonomous driving mode before passing through the tolling station are the same), the present routine proceeds to step S208.

In step S207, the gate selecting unit 16 utilizes the selection criterion 2 to select a gate from among the plurality of gates provided at the tolling station. That is, a gate which enables a smooth transition between different autonomous driving modes is selected as the gate for the own vehicle V to pass through. After the processing in step S207, the present routine proceeds to step S205.

In step S208, the gate selecting unit 16 utilizes the selection criterion 3 to select a gate from among the plurality of gates provided at the tolling station. That is, a gate through which the own vehicle V can arrive at the destination earliest is selected as the gate for the own vehicle V to pass through. After the processing in step S208, the present routine proceeds to step S205.

When the driving performed until the own vehicle V passes through the tolling station is autonomous driving and the driving performed after passing through the tolling station is manual driving, step S209 is selected. In step S209, the gate selecting unit 16 performs a conditional judgement to judge if the preparations of the driver (preparations to receive handover of the driving operation) are not completed. This conditional judgement is performed based on the driver state acquired in step S1 of the main routine. If the result of the conditional judgement is “Yes” (when the preparations of the driver are not completed), the present routine proceeds to step S210. If the result of the conditional judgement is “No” (when the preparations of the driver are completed), the present routine proceeds to step S211.

In step S210, the gate selecting unit 16 utilizes the selection criterion 1 to select a gate from among the plurality of gates provided at the tolling station. That is, a gate which enables the driving operation to be safely handed over to the driver is selected as the gate for the own vehicle V to pass through. After the processing in step S210, the present routine proceeds to step S205.

In step S211, the gate selecting unit 16 utilizes the selection criterion 3 to select a gate from among the plurality of gates provided at the tolling station. That is, a gate through which the own vehicle V can arrive at the destination earliest is selected as the gate for the own vehicle V to pass through. After the processing in step S211, the present routine proceeds to step S205.

In step S205, the gate selecting unit 16 outputs the gate selected in step S204, S207, S210 or S211 to the path and speed plan creation unit 17 and the gate presentation unit 19. The path and speed plan creation unit 17 reflects the gate selected by the gate selecting unit 16 in the path and speed plan for autonomous driving. The gate presentation unit 19 presents the gate selected by the gate selecting unit 16 to the driver through the HMI 7.

If the driving to be performed until the own vehicle V passes through the tolling station is manual driving, step S212 is selected. In step S212, the gate selecting unit 16 utilizes the selection criterion 3 to select a gate from among the plurality of gates provided at the tolling station. That is, a gate through which the own vehicle V can arrive at the destination earliest is selected as the gate for the own vehicle V to pass through. After the processing in step S212, the present routine proceeds to step S213.

In step S213, the gate selecting unit 16 outputs the gate selected in step S212 to the gate presentation unit 19. The gate presentation unit 19 presents the gate that the gate selecting unit 16 selected to the driver through the HMI 7, to suggest the gate to which the own vehicle V should proceed to the driver.

### 2-3-4. Details of Sub-Routine 3

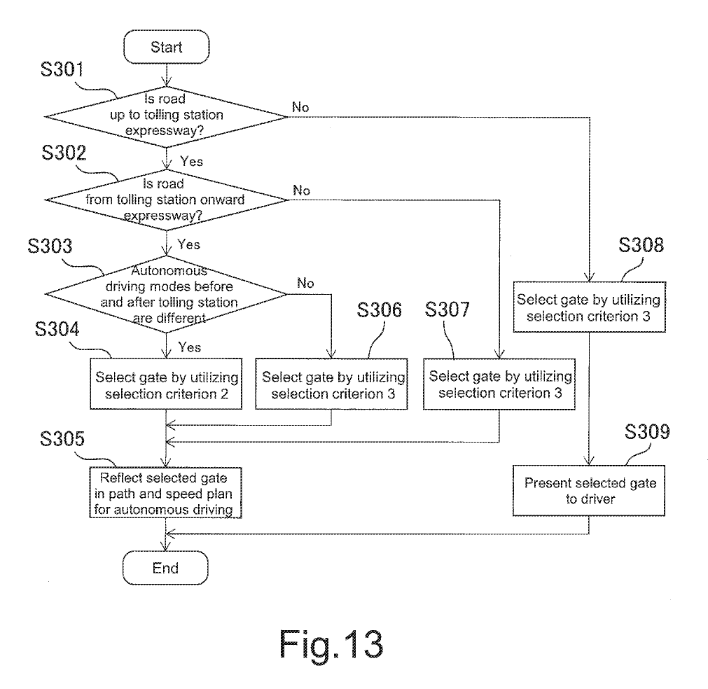

The sub-routine 3 is called in the main routine when a road that the own vehicle V travels on until passing through the tolling station is an ordinary road. FIG. 13 is a view in which the sub-routine 3 is represented by a flowchart. Hereunder, the contents of each step constituting the sub-routine 3 will be described in order from step S301.

In step S301, the gate selecting unit 16 performs a conditional judgement to judge whether driving that will be performed until the own vehicle V passes through the tolling station is autonomous driving or manual driving. This conditional judgement is performed based on the autonomous driving utilization determination performed in step S2 of the main routine. If the result of the conditional judgement is “Yes” (when the driving performed until the own vehicle V passes through the tolling station is autonomous driving), the present routine proceeds to step S302. If the result of the conditional judgement is “No” (when the driving performed until the own vehicle V passes through the tolling station is manual driving), the present routine proceeds to step S308.

In step S302, the gate selecting unit 16 performs a conditional judgement to judge whether driving that will be performed after the own vehicle V passes through the tolling station is autonomous driving or manual driving. This conditional judgement is performed based on the autonomous driving utilization determination performed in step S2 of the main routine. If the result of the conditional judgement is “Yes” (when the driving that will be performed after the own vehicle V passes through the tolling station is autonomous driving), the present routine proceeds to step S303. If the result of the conditional judgement is “No” (when the driving that will be performed after the own vehicle V passes through the tolling station is manual driving), the present routine proceeds to step S307.

In step S303, the gate selecting unit 16 performs a conditional judgement to judge whether or not the autonomous driving mode utilized after passing through the tolling station will be different from the autonomous driving mode utilized before passing through the tolling station. This conditional judgement is performed based on the autonomous driving utilization determination performed in step S2 of the main routine. If the result of the conditional judgement is “Yes” (when the autonomous driving mode after passing through the toiling station is different from the autonomous driving mode before passing through the toiling station), the present routine proceeds to step S304. If the result of the conditional judgement is “No” (when the autonomous driving mode after passing through the tolling station and the autonomous driving mode before passing through the tolling station are the same), the present routine proceeds to step S306.

In step S304, the gate selecting unit 16 utilizes the selection criterion 2 to select a gate from among the plurality of gates provided at the tolling station. That is, a gate which enables a smooth transition between different autonomous driving modes is selected as the gate for the own vehicle V to pass through. After the processing in step S304, the present routine proceeds to step S305.

In step S306, the gate selecting unit 16 utilizes the selection criterion 3 to select a gate from among the plurality of gates provided at the tolling station. That is, a gate through which the own vehicle V can arrive at the destination earliest is selected as the gate for the own vehicle V to pass through. After the processing in step S306, the present routine proceeds to step S305.

When the driving performed until the own vehicle V passes through the tolling station is autonomous driving and the driving performed after passing through the tolling station is manual driving, step S307 is selected. In step S307, the gate selecting unit 16 utilizes the selection criterion 3 to select a gate from among the plurality of gates provided at the tolling station. That is, a gate through which the own vehicle V can arrive at the destination earliest is selected as the gate for the own vehicle V to pass through. After the processing in step S307, the present routine proceeds to step S305.

In step S305, the gate selecting unit 16 outputs the gate selected in step S304, S306 or S307 to the path and speed plan creation unit 17 and the gate presentation unit 19. The path and speed plan creation unit 17 reflects the gate selected by the gate selecting unit 16 in the path and speed plan for autonomous driving. The gate presentation unit 19 presents the gate selected by the gate selecting unit 16 to the driver through the HMI 7.

If the driving to be performed until the own vehicle V passes through the tolling station is manual driving, step S308 is selected. In step S308, the gate selecting unit 16 utilizes the selection criterion 3 to select a gate from among the plurality of gates provided at the tolling station. That is, a gate through which the own vehicle V can arrive at the destination earliest is selected as the gate for the own vehicle V to pass through. After the processing in step S308, the present routine proceeds to step S309.

In step S309, the gate selecting unit 16 outputs the gate selected in step S308 to the gate presentation unit 19. The gate presentation unit 19 presents the gate selected by the gate selecting unit 16 to the driver through the HMI 7, to thereby suggest to the driver the gate to which to proceed.

### 3. Processing, for Transitioning Between Different Autonomous Driving Modes

In the case of switching from one autonomous driving mode to another autonomous driving mode at a tolling station, because differences with respect to identification and with respect to the control structure exist between different autonomous driving modes, there is a possibility that behavior which is not smooth will occur in the own vehicle V. To suppress the occurrence of such a situation, selection of a gate utilizing the selection criterion 2 is performed by the gate selecting unit 16 as described above. Further, according to the present embodiment, in addition to the selection of a gate utilizing the selection criterion 2, the following processing is performed by the path and speed plan creation unit 17.

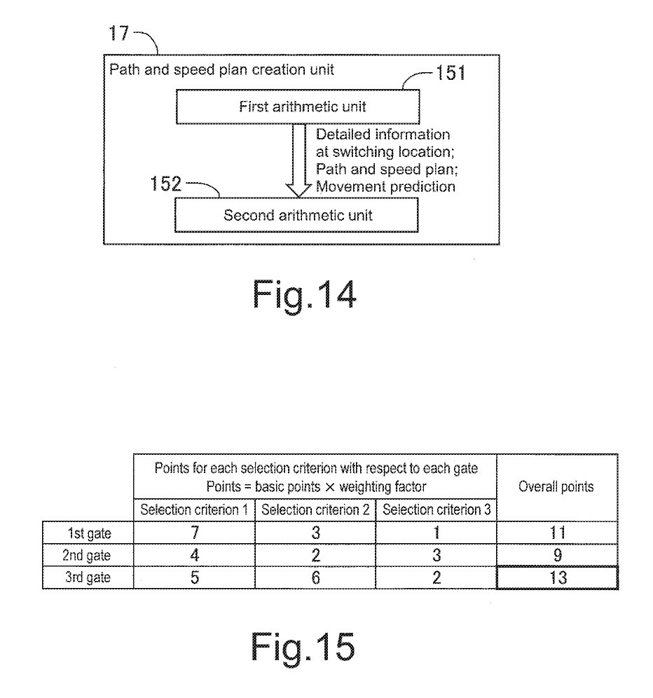

FIG. 14 is a block diagram illustrating the configuration of the path and speed plan creation unit 17. The path and speed plan creation unit 17 includes a first arithmetic unit 151 and a second arithmetic unit 152. The first arithmetic unit 151 creates a path and speed plan for the own vehicle V up to the tolling station in accordance with the autonomous driving mode that is selected in the section up to the tolling station. The second arithmetic unit 152 creates a path and speed plan for the own vehicle V from the tolling station onward in accordance with the autonomous driving mode that is selected for the section from the tolling station onward.

At a switching location where the autonomous driving modes are switched, the first arithmetic unit 151 passes detailed information for the status at the switching location to the second arithmetic unit 152. The detailed information includes positional information, the yaw angle, the speed, the acceleration, the steering angle and the angular velocity of the steering angle of the own vehicle V. Further, the first arithmetic unit 151 passes to the second arithmetic unit 152 a path plan (specifically, coordinates of passing points on the path) and a speed plan (specifically, transitions in the speed on the path) until the switching location from a position that is a predetermined distance (for example, several dozen meters) before the switching location. In addition, the first arithmetic unit 151 passes to the second arithmetic unit 152 a movement prediction with respect to objects (vehicles and people) around the own vehicle V. This is because if the movement prediction with respect to objects around the switching location changes, a sudden change is liable to arise in the behavior of the own vehicle V.

The second arithmetic unit 152 uses the above described information passed thereto from the first arithmetic unit 151 to create a path and speed plan that does not cause sudden changes to arise in the acceleration, steering angle and angular velocity of the steering angle of the own vehicle V. By performing this processing at the path and speed plan creation unit 17, by means of a synergistic effect obtained with the gate selection by the gate selecting unit 16, the occurrence of behavior that is not smooth in the own vehicle V when the autonomous driving mode is switched can be suppressed.

### 4. Other Embodiments

Hereinafter, other embodiments will be described,

### 4-1. Modification of Gate Selection Procedures

In the rules for determining the selection criteria shown in the table in FIG. 7, when the driving performed on an expressway is autonomous driving and driving performed on an ordinary road is also autonomous driving, the selection criteria that are utilized are the selection criteria 1, 2 and 3. In the above described embodiment, as shown in the flowchart in FIG. 12, any one of the selection criteria 1, 2 and 3 is exclusively utilized. However, as described hereunder, selection of a gate can also be performed by simultaneously utilizing all of the selection criteria 1, 2 and 3.

FIG. 15 is a table for describing procedures for selecting a gate by simultaneously utilizing all of the selection criteria. In the table, points are given to each selection criterion with respect to three gates from a first gate to a third gate, respectively. More specifically, basic points are given to each selection criterion for each gate according to the degree (level of compatibility) to which the gate satisfies the selection criterion, and a weighting factor is assigned to the basic points according to the degree to which the selection criterion is given priority (a priority level) over other selection criteria. The points in the table are evaluation points for each selection criterion with respect to each gate that are obtained by multiplying the basic points by the weighting factor. The number of evaluation points increases as the level of compatibility of a gate to a selection criterion increases, or as the priority level of the selection criterion increases.

The level of compatibility of a gate to a selection criterion is not necessarily fixed. For example, the level of compatibility of each gate to the selection criterion 3 changes according to the relation between the target route and the road shape after passing through the tolling station, and changes according to the degree of crowding at the tolling station. Further, the priority level of a selection criterion changes due to various factors. For example, the priority level of the selection criterion 1 decreases when the level of vigilance of a driver is high, and increases when the level of vigilance of a driver is low. The priority level of the selection criterion 3 decreases if there is no delay relative to the traveling plan, and increases as a delay relative to the traveling plan increases. Hence, the evaluation points for each selection criterion with respect to each gate that are determined based on the level of compatibility and the priority level are variable values that change according to the circumstances.

In the table shown in FIG. 15, overall points that are the combined (in this case, totaled) evaluation points for the respective selection criteria are described for each of the three gates from the first gate to the third gate. The overall points represent the evaluation for each gate, and the gate with the highest evaluation score is the optimal gate to cause the own vehicle V to pass through. In the example illustrated in FIG. 15, since the overall points of the third gate is the highest at 13 points, the third gate is selected as the gate for the own vehicle V to pass through.

### 4-1-1. Modification of Sub-Routine 2

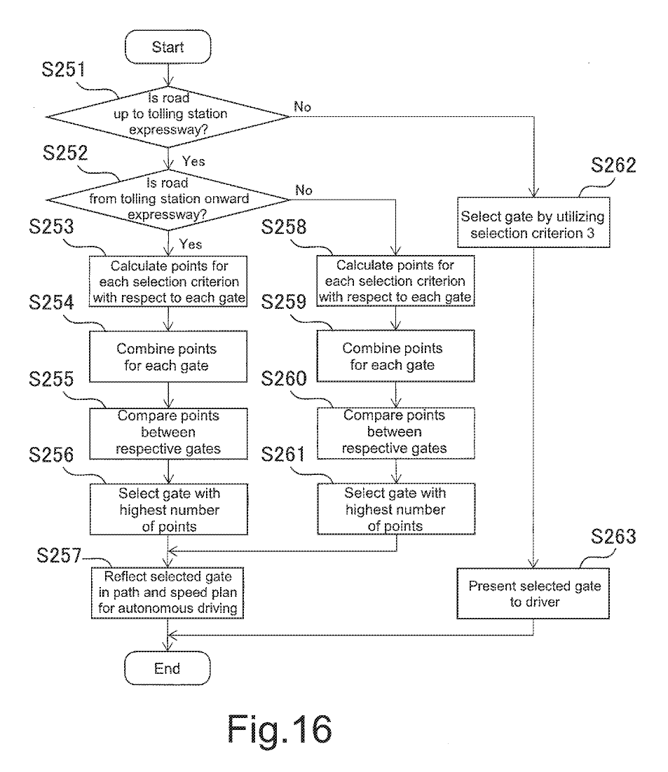

When the gate selection procedures described above are applied to the gate selection program of the above embodiment, the sub-routine 2 constituting part of the gate selection program is modified as shown in the flowchart in FIG. 16. Hereunder, the contents of each step constituting the modification of the sub-routine 2 are described in order from step S251.

In step S251, the gate selecting unit 16 performs a conditional judgement to judge whether driving to be performed until the own vehicle V passes through the tolling station is autonomous driving or manual driving. This conditional judgement is performed based on the autonomous driving utilization determination performed in step S2 of the main routine. If the result of the conditional judgement is “Yes” (when the driving to be performed until the own vehicle V passes through the tolling station is autonomous driving), the present routine proceeds to step S252. If the result of the conditional judgement is “No” (when the driving to be performed until the own vehicle V passes through the tolling station is manual driving), the present routine proceeds to step S262.

In step S252, the gate selecting unit 16 performs a conditional judgement to judge whether driving that will be performed after the own vehicle V passes through the tolling station is autonomous driving or manual driving. This conditional judgement is performed based on the autonomous driving utilization determination performed in step S2 of the main routine. If the result of the conditional judgement is “Yes” (when the driving that will be performed after the own vehicle V passes through the tolling station is autonomous driving), the present routine proceeds to step S253, If the result of the conditional judgement is “No” (when the driving that will be performed after the own vehicle V passes through the tolling station is manual driving), the present routine proceeds to step S258.

If the driving to be performed until the own vehicle V passes through the tolling station is autonomous driving, and the driving to be performed after passing through the tolling station is autonomous driving, the selection criteria 1, 2 and 3 are utilized as shown in the table in FIG. 7. In step S253, with respect to the selection criteria 1, 2 and 3, the gate selecting unit 16 calculates evaluation points for each selection criterion for the respective gates.

Next, in step S254, for the respective gates, the gate selecting unit 16 combines the evaluation points for each selection criterion for each gate that were calculated in step S253, to thereby calculate the overall points for the respective gates.

Subsequently, in step S255, the gate selecting unit 16 performs a comparison of the overall points for each gate calculated in step S254 between the respective gates.

Next, in step S256, the gate selecting unit 16 selects the gate with the highest number of points in the comparison result of step S255 as the gate for the own vehicle V to pass through. After the processing in step S256, the present routine proceeds to step S257.

If the driving to be performed until the own vehicle V passes through the tolling station is autonomous driving, and the driving to be performed after passing through the tolling station is manual driving, the selection criteria 1 and 3 are utilized as shown in the table in FIG. 7. In step S258, with respect to the selection criteria 1 and 3, the gate selecting unit 16 calculates evaluation points for each selection criterion for the respective gates.

Next, in step S259, for the respective gates, the gate selecting unit 16 combines the evaluation points for each selection criterion for each gate that were calculated in step S258, to thereby calculate the overall points for the respective gates.

Subsequently, in step S260, the gate selecting unit 16 performs a comparison of the overall points for each gate calculated in step S259 between the respective gates.

Next, in step S261, the gate selecting unit 16 selects the gate with the highest number of points in the comparison result of step S260 as the gate for the own vehicle V to pass through. After the processing in step S260, the present routine proceeds to step S257.

In step S257, the gate selecting unit 16 outputs the gate selected in step S256 or S261 to the path and speed plan creation unit 17 and the gate presentation unit 19. The path and speed plan creation unit 17 reflects the gate selected by the gate selecting unit 16 in the path and speed plan for autonomous driving. The gate presentation unit 19 presents the gate selected by the gate selecting unit 16 to the driver through the HMI 7.

If the driving to be performed until the own vehicle V passes through the tolling station is manual driving, step S262 is selected. In step S262, the gate selecting unit 16 utilizes the selection criterion 3 to select a gate from among the plurality of gates provided at the tolling station. After the processing in step S262, the present routine proceeds to step S263.

In step S263, the gate selecting unit 16 outputs the gate selected in step S262 to the gate presentation unit 19. The gate presentation unit 19 presents the gate selected by the gate selecting unit 16 to the driver through the HMI 7, to thereby suggest to the driver the gate to which to proceed.

What is claimed is:

1. A path determination apparatus that is mounted in a vehicle which is capable of autonomous driving, comprising;
at least one processor; and
at least one memory including at least one computer program, the at least one memory and the at least one computer program configured, with the at least one processor, to cause the path determination apparatus at least to operate as:
a gate selecting unit that, when the vehicle passes through a tolling station having a plurality of gates, selects a gate to pass through from among the plurality of gates,
wherein the gate selecting unit is configured to make a judgement as to whether or not a road that the vehicle travels on until passing through the tolling station is an expressway, as one conditional judgement for selecting the gate to pass through.

2. The path determination apparatus according to claim 1,
wherein the gate selecting unit is configured to make a judgement as to whether or not a road that the vehicle is to travel on after passing through the tolling station is an expressway, as one conditional judgement for selecting the gate to pass through.

3. The path determination apparatus according to claim 1,
wherein the gate selecting unit is configured to make a judgement as to whether driving that is performed until the vehicle passes through the tolling station is autonomous driving or manual driving, as one conditional judgement for selecting the gate to pass through.

4. The path determination apparatus according to claim 3,
wherein the gate selecting unit is configured to make a judgement as to whether driving to be performed after the vehicle passes through the tolling station is autonomous driving or manual driving, as one conditional judgement for selecting the gate to pass through.

5. The path determination apparatus according to claim 1,
wherein the gate selecting unit is configured to define a plurality of selection criteria in advance, determine a selection criterion for selecting a gate to be passed through from among the plurality of selection criteria in accordance with the conditional judgement, and select a gate that most satisfies the determined selection criterion from among the plurality of gates.

6. The path determination apparatus according to claim 5,
wherein the gate selecting unit is configured to, when there is a plurality of selection criteria which correspond to a result of the conditional judgement, evaluate each of the plurality of gates based on a priority level of each selection criterion and a level of compatibility of the plurality of gates with each selection criterion, and select a gate for which an evaluation is highest.
